(19) United States
(12) Patent Application Publication
(10) Pub. No.: US 2005/0176649 A1
(43) Pub. Date: Aug. 11, 2005

(54) Iap binding peptides and assays for identifying compounds that bind iap

(76) Inventors: George McLendon, Princeton, NJ (US); Rachel A. Kipp, Plainsboro, NJ (US); Martin Case, Princeton, NJ (US); Yigong Shi, Pennington, NJ (US)

Correspondence Address: Janet E Reed;Woodcock Washburn, 46th Floor, One Liberty Place, Philadelphia, PA, 19103, US

(21) Appl. No.: 10/478,521

(22) PCT Filed: May 31, 2002
(86) PCT No.: PCT/US02/17342

Related U.S. Application Data
(60) Provisional application No. 60/294,682, filed on May 31, 2001.
(60) Provisional application No. 60/345,630, filed on Jan. 3, 2002.

(51) Int. Cl. A61K 38/05; C07K 5/06

(52) U.S. Cl. 514/18; 530/331

(57) ABSTRACT

Assays are disclosed for identifying peptides and peptidomimetics for promoting apotosis in cells, through a pathway involving the Inhibitor of Apoptosis Proteins (IAPs), exemplified by XIAP, and the mitochondrial protein Smac/DIABOLO (hereinafter Smac) and homologs thereof. Also disclosed are IAP-binding peptides and peptidomimetics identified through the use of the assay.

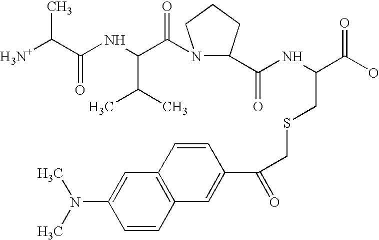

This application claims benefit of U.S. Provisional Application Nos. 60/294,682, filed May 31, 2001, and 60/345,630, filed Jan. 3, 2002, the entirety of each of which is incorporated by reference herein.

Pursuant to 35 U.S.C. §202(c), it is acknowledged that the U.S. Government has certain rights in the invention described herein, which was made in part with funds from the National Institutes of Health, Grant No. GM59348-02.

### FIELD OF THE INVENTION

The present invention relates to the field of drug design and development for prevention and treatment of cell proliferative disease. Specifically, the invention features an assay for identifying peptides and peptidomimetics for promoting apotosis in cells, through a pathway involving the Inhibitor of Apoptosis Proteins (IAPs), exemplified by XIAP, and the mitochondrial protein Smac/DIABOLO (hereinafter Smac). The invention also features peptides and peptidomimetics identified through the use of the assay.

### BACKGROUND OF THE INVENTION

Various scientific articles, patents and other publications are referred to throughout the specification. Each of these publications is incorporated by reference herein in its entirety.

Apoptosis (programmed cell death) plays a central role in the development and homeostasis of all multi-cellular organisms. Alterations in apoptotic pathways have been implicated in many types of human pathologies, including developmental disorders, cancer, autoimmune diseases, as well as neuro-degenerative disorders.

Thus, the programmed cell death pathways have become attractive targets for development of therapeutic agents. In particular, since it is conceptually easier to kill than to sustain cells, attention has been focused on anti-cancer therapies using pro-apoptotic agents such as conventional radiation and chemo-therapy. These treatments are generally believed to trigger activation of the mitochondria-mediated apoptotic pathways. However, these therapies lack molecular specificity, and more specific molecular targets are needed.

Apoptosis is executed primarily by activated caspases, a family of cysteine proteases with aspartate specificity in their substrates. Caspases are produced in cells as catalytically inactive zymogens and must be proteolytically processed to become active proteases during apoptosis. In normal surviving cells that have not received an apoptotic stimulus, most caspases remain inactive. Even if some caspases are aberrantly activated, their proteolytic activity can be fully inhibited by a family of evolutionarily conserved proteins called IAPs (inhibitors of apoptosis proteins) (Deveraux & Reed, Genes Dev. 13: 239-252, 1999). Each of the IAPs contains 1-3 copies of the so-called BIR (baculoviral IAP repeat) domain and directly interacts with and inhibits the enzymatic activity of mature caspases. Several distinct mammalian IAPs including XIAP, survivin, and Livin/ML-IAP (Kasof & Gomes, J. Biol. Chem. 276: 3238-3246, 2001; Vucic et al. Curr. Biol. 10: 1359-1366, 2000; Ashhab et al. FEBS Lett. 495: 56-60, 2001), have been identified, and they all exhibit anti-apoptotic activity in cell culture (Deveraux & Reed, 1999, supra). As IAPs are expressed in most cancer cells, they may directly contribute to tumor progression and subsequent resistance to drug treatment.

In normal cells signaled to undergo apoptosis, however, the LAP-mediated inhibitory effect must be removed, a process at least in part performed by a mitochondrial protein named Smac (second mitochondria-derived activator of caspases; Du et al. Cell 102: 33-42, 2000) or DIABLO (direct IAP binding protein with low pI; Verhagen et al. Cell 102: 43-53, 2000). Smac, synthesized in the cytoplasm, is targeted to the inter-membrane space of mitochondria. Upon apoptotic stimuli, Smac is released from mitochondria back into the cytosol, together with cytochrome c. Whereas cytochrome c induces multimerization of Apaf-1 to activate procaspase-9 and -3, Smac eliminates the inhibitory effect of multiple IAPs. Smac interacts with all IAPs that have been examined to date, including XIAP, c-IAP1, c-IAP2, and survivin (Du et al., 2000, supra; Verhagen et al., 2000, supra). Thus, Smac appears to be a master regulator of apoptosis in mammals.

Smac is synthesized as a precursor molecule of 239 amino acids; the N-terminal 55 residues serve as the mitochondria targeting sequence that is removed after import (Du et al., 2000, supra). The mature form of Smac contains 184 amino acids and behaves as an oligomer in solution (Du et al., 2000, supra). Smac and various fragments thereof have been proposed for use as targets for identification of therapeutic agents. U.S. Pat. No. 6,110,691 to Wang et al. describes the Smac polypeptide and fragments ranging from at least 8 amino acid residues in length. However, the patent neither discloses nor teaches a structural basis for choosing a particular peptide fragment of Smac for use as a therapeutic agent or target.

Similar to mammals, flies contain two IAPs, DLAP1 and DIAP2, that bind and inactivate several Drosophila caspases (Hay, Cell Death Differ. 7: 1045-1056, 2000). DIAP1 contains two BIR domains; the second BIR domain (BIR2) is necessary and sufficient to block cell death in many contexts. In Drosophila cells, the anti-death function of DIAP1 is removed by three pro-apoptotic proteins, Hid, Grim, and Reaper, which physically interact with the BIR2 domain of DIAP1 and remove its inhibitory effect on caspases. Thus Hid, Grim, and Reaper represent the functional homologs of the mammalian protein Smac. However, except for their N-terminal 10 residues, Hid, Grim, and Reaper share no sequence homology with one another, and there is no apparent homology between the three Drosophila proteins and Smac.

In commonly-owned co-pending application Ser. No. 09/965,967 (the entirety of which is incorporated by reference herein), it is disclosed that the above described biological activity of Smac is related to binding of its N-terminal four residues to a featured surface groove in a portion of XIAP referred to as the BIR3 domain. This binding prevents XIAP from exerting its apoptosis-suppressing function in the cell. It was further disclosed that N-terminal tetrapeptides from LAP binding proteins of the Drosophila pro-apoptotic proteins Hid, Grim and Veto function in the same manner.

The development of apoptosis-promoting therapeutic agents based on the IAP-binding peptide of Smac or its homologs from other species would be greatly facilitated by high throughput screening assays to identify useful molecules. Further, development of such therapeutic agents would be accelerated by the production of libraries of rationally designed candidate compounds.

### SUMMARY OF THE INVENTION

The present invention features an assay for use in high throughput screening or rational drug design of agents that can, like the Smac tetrapeptide or its homologs in other species, bind to a BIR domain of an IAP, thereby relieving IAP-mediated suppression of apoptosis. These assays make use of the discoveries made in accordance with the invention disclosed in commonly-owned, co-pending U.S. application Ser. No. 09/965,967 that (1) the N-terminal tetrapeptide motif of Smac and other IAP binding proteins is sufficient for binding to IAPs and (2) the mammalian BIR3 domain and the Drosophila BIR2 domain comprise a specific binding groove for the tetrapeptide.

The assay comprises the following basic steps: (a) providing a labeled mimetic of an IAP-binding tetrapeptide that binds to the appropriate BIR domain (preferably BIR3), wherein at least one measurable feature of the label changes as a function of the mimetic being bound to the IAP or free in solution; (b) contacting the BIR domain of an IAP with the labeled mimetic under conditions enabling binding of the mimetic to the BIR domain, thereby forming a BIR-labeled mimetic complex having the measurable feature; (c) contacting the BIR-labeled mimetic complex with the compound to be tested for BIR binding; and (d) measuring displacement of the labeled mimetic from the BIR-labeled mimetic complex, if any, by the test compound, by measuring the change in the measurable feature of the labeled mimetic, thereby determining if the test compound is capable of binding to the LAP. In a preferred embodiment, the labeled mimetic is AVPX (SEQ ID NO:1), wherein X is directly or indirectly linked to a fluorigenic dye. Preferably, it is AVPC (SEQ ID NO:2) attached to a badan dye.

The present invention also provides a library of peptides or peptidomimetics that have been demonstrated by the methods of the invention to bind to the BIR3 domain of XIAP. In one embodiment, these peptides are composed of naturally-occurring amino acid residues. In another embodiment, the library is based on a peptidomimetic, which may be partially or fully non-peptide in nature, but which mimics the physicochemical features of the Smac peptide such that it is capable of binding IAP.

### BRIEF DESCRIPTION OF THE DRAWINGS

FIG. 1 shows the chemical structure of AVPC-badan dye.

FIG. 2 shows absorption and emission properties of AVPC-badan. FIG. 2A shows the absorption (solid line) and emission (dotted line) spectra of the molecule in water. FIG. 2B shows the solvatochromicity of AVPC-badan in acetonitrile (ACN), with respect to the emission spectrum.

FIG. 3 shows the emission spectra of AVPC-badan in the presence of BIR3 at different concentrations of BIR3. Measurements were taken in 50 mM Tris buffer, pH 7.1, 100 mM NaCL, 2 mM DTT and 5.1 μM badan dye, excitation wavelength 387 nm.

FIG. 4 shows emission spectra of samples from the binding assay described in the text, the results of which are shown in Table 2. All samples were 5 μM in both dye and protein, and 50 mM in the tetrapeptide. The buffer was 50 mM Tris at pH 7.1, 100 mM NaCl and 2 mM DTT. The AVPI (SEQ ID NO:3) tetrapeptide displayed was synthesized separately from the other samples.

FIG. 5 shows (A) absorption (−) and emission ( - - - ) spectra of AVPC-badan in water (excitation at 387 nm) (These spectra are also shown in FIG. 2); and (B) titration of AVPC-badan with BIR3. The fraction of free AVPC-badan was determined by relating the difference of the observed fluorescence intensity and a maximum intensity where all of the dye is assumed to be bound, Iᵥ, to the difference between the intensity of the unbound dye and Iᵥ. Data are discussed in Example 1.

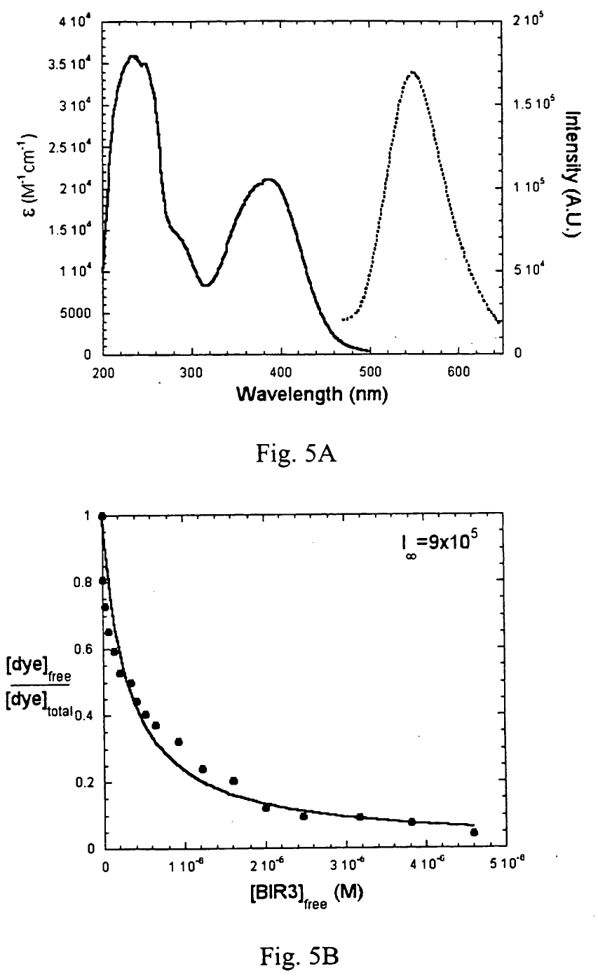

FIG. 6 shows (A) emission spectra of AVPC-badan, AVPC-badan in the presence of BIR3 and AVPF (SEQ ID NO:4), AVPC-badan in the presence of BIR3 and ARPI (SEQ ID NO:5), AVPC-badan in the presence of BIR3 and AVPI (SEQ ID NO:3), AVPC-badan in the presence of BIR3 and GVPI (SEQ ID NO:6), AVPC-badan in the presence of BIR3 and AGPI (SEQ ID NO:7), and AVPC-badan in the presence of BIR3, in order of increasing emission intensity; and (B) correlation of hydrophobic interaction expressed as ΔG₁ (EtOH—H₂O) (23) with ΔG_{b} for a range of nonpolar amino acids (polar amino acids are not shown in this graph). Data are discussed in Example 1.

### DETAILED DESCRIPTION OF THE INVENTION

The ability to quickly assay small molecules for their effectiveness in disrupting protein-protein interactions is critical to the development of viable drug candidates. One aspect of the present invention comprises an assay to test the binding affinity of a library of tetrapeptide molecules for the BIR3 domain of an inhibitor of apoptosis protein (LAP), particularly the mammalian XIAP. The assay is based on a detectable label, preferably a fluorogenic dye molecule. In preferred embodiments, the fluorophore is attached to a tripeptide, AVP, whose sequence matches the N-terminal three residues of Smac. The general structure of this molecule, therefore, is AVP[X], wherein X is the fluorophore. The molecule is referred to herein as an “AVP-dye”. The AVP-dye packs into the groove of the BIR3, causing a large shift in emission maximum and intensity when the environment of the dye changes from water to the hydrophobic pocket of the protein. If a molecule (e.g. the native Smac protein or a tetrapeptide mimic) displaces the dye, then emission will shift back to the spectrum observed in water. Since the emission intensity is related to the binding of the tetrapeptide, the intensity can be used to estimate the equilibrium constant, K, for displacement of the AVP-dye by the tetrapeptide. The larger the equilibrium constant, the greater affinity the tetrapeptide has for the BIR3. This allows the most promising inhibitors to be quickly determined, and structural information about effective inhibitors can be incorporated into the design of candidates for the next round of testing.

It will be understood by those of skill in the art that, though the AVP dye-BIR3 system described above is exemplified and preferred for practice of the invention, various combinations of (1) LAP-binding tetrapeptides and mimetics, (2) BIR binding grooves and (3) detectable labels may be used interchangeably to create variations of the assay described above. Particular reference is given to the consensus tetrapeptide set forth in co-pending U.S. application Ser. No. 09/965,967, which is A-(V/T/I)-(P/A)-(F/Y/I/V) (SEQ ID NO:8).

Without intending to be limited by any explanation as to mechanism, it is believed that the underlying factors influencing binding of the labeled tetrapeptide AVP-dye to the BIR binding groove include the following:
1. Recognition is achieved through hydrogen bond interactions and van der Waals contacts.
2. Eight inter- and three intra-molecular hydrogen bonds support the binding of AVPI in the surface groove on BTR3.
3. Three intermolecular contacts between the backbone groups of Val2/Ile4 in Smac and Gly306/Thr308 in BIR3 allow the formation of a 4 stranded antiparallel β sheet.
4. Ala1 donates 3 hydrogen bonds to Glu314 and Gln319, and its carbonyl makes contact with Gln319 and Trp323.
5. The methyl group of Ala1 fits tightly in a hydrophobic pocket formed by the side chains of Leu307, Trp310, and Gln319.
6. Val2 and Pro3 maintain multiple van der Waals interactions with Trp323, and Pro3 has an additional interaction with Tyr324.
7. The side chain of Ile4 interacts with Leu292, Gly306, Lys297 and Lys299.

Accordingly, the AVP-dye may comprise any suitable detectable label, such as a fluorophore, such that binding of the label does not detrimentally affect binding of the dye to the BIR3, via any one or more of the foregoing factors. A particularly suitable dye for use in the AVP-dye is 6-Bromoacetyl-2-dimethylaminonaphthalene (badan) dye. Badan is a fluorogenic dye whose sensitivity to environmental changes has previously been made use of to probe protein binding interactions (Boxrud et al. J. Biol. Chem. 275: 14579-14589, 2000; Owenius et al., Biophys. J. 77: 2237-2250, 1999; Hiratsuka, T. J. Biol. Chem. 274: 29156-29163, 1999).

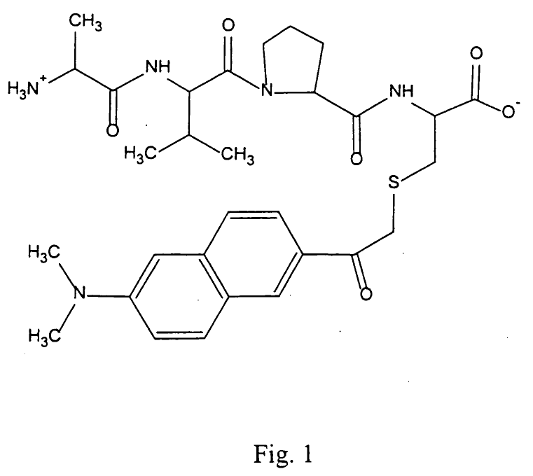

The synthesis of NH₃⁺-AVPC(badan)amide is described below, and its chemical structure is shown in FIG. 1. Unless otherwise stated, materials were purchased from Aldrich Chemical Co. (Milwaukee, Wis.) or Fisher Scientific (Pittsburgh, Pa.) and used without further purification. Methylbenzhydrylamine (MBHA) solid-phase peptide synthesis resin and Fmoc amino acids were obtained from Advanced ChemTech (Louisville, Ky.) and NovaBiochem (San Diego, Calif.). Badan dye was obtained from Molecular Probes (Eugene, Oreg.).

The peptide was synthesized on a hand shaker by Fmoc protocol on MBHA resin (Chan, W. C.; White, P. D. Fmoc Solid Phase Peptide Synthesis: A Practical Approach; Oxford University Press: Oxford, 2000). The MBHA resin was chosen because the protocol requires that it be stable under both acidic and basic conditions. The Ala-Val-Pro-Cys peptide was synthesized using a trityl group to protect the Cysteine thiol. Prior to the deprotection of the Fmoc group of the alanine, the trityl group was removed by the addition of trifluoroacetic acid (TFA), and the cysteine was derivatized with badan in the presence of diisopropylethylamine (DIEA). The Fmoc group of the alanine was removed with piperidine and then cleavage from the resin was effected by treatment with anhydrous HF containing 10% v/v anisole as scavenger at 0° C. for 45 minutes. The labeled peptide was purified by HPLC on a Vydac C18 preparative column with gradient elution by solvents A (99% H₂O; 1% CH₃CN; 0.1% TFA) and B (90% CH₃CN; 10% H₂O; 0.1% TFA) and lyophilized to dryness prior to reconstitution in H₂O.

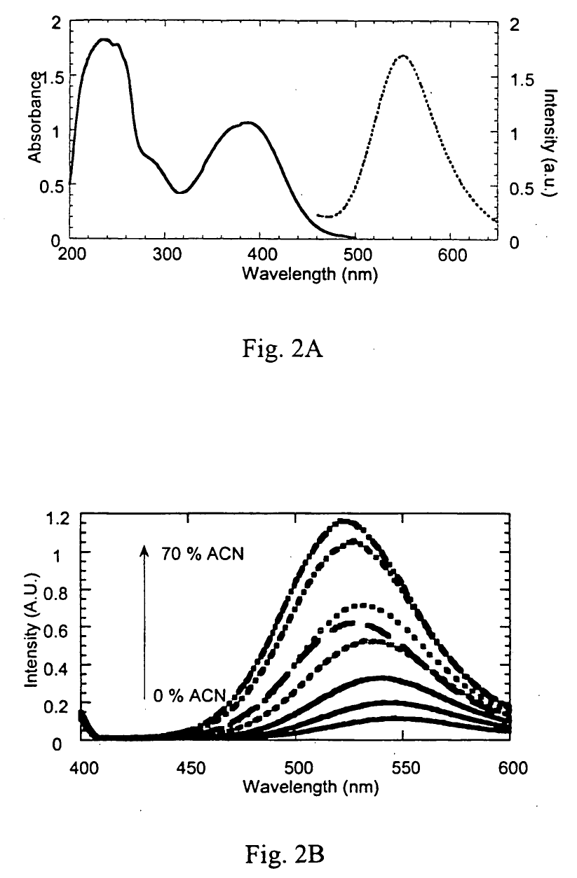

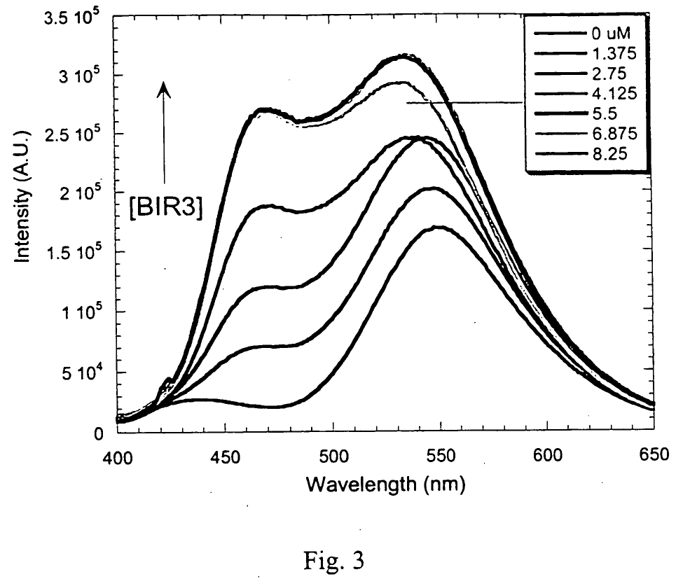

Absorption and emission properties of AVPC-badan are shown in FIG. 2. FIG. 2A shows the absorption and emission spectra of the molecule in water. FIG. 2B shows the solvatochromicity of AVPC-badan in acetonitrile (ACN), with respect to the emission spectrum. FIG. 3 shows the emission spectra of AVPC-badan in the presence of BIR3 at different concentrations of BIR3.

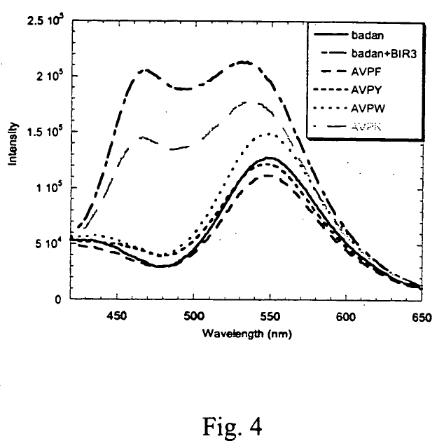

The aforementioned AVP-dye is used in an assay of test compounds that may, like the Smac tetrapeptide AVPI, bind to the BIR3 domain of XIAP, thereby relieving XIAP-mediated suppression of apoptosis. This is a high-throughput, cell-free assay, that is assembled as follows. A protein comprising the BIR3 domain of an IAP is placed in an assay medium comprising a suitable buffer, as described above. Preferably, this is a recombinant protein comprising the BIR3 domain, but a full IAP protein also may be used. An aliquot of the AVP-dye is added to the reaction mixture, in the presence of the test compound. Controls comprise the BIR3 and the dye in the absence of the test compound and, optionally, BIR3 and the dye in the presence of the naturally occurring tetrapeptide, AVPI. The fluorescence of the reaction mixture at a selected excitation and emission wavelength, e.g., 387 nm excitation, 545 nm emission, is measured. Alternatively, a emission spectrum is measured at the selected excitation wavelength. In one type of measurement, the test compound is added and an emission spectrum is measured by scanning from, e.g., 460-480 nm. In another type of measurement, the emission intensity at a particular wavelength, e.g., 470 nm, is measured. The emission spectrum of the dye bound to BIR3 is distinctly different from the spectrum of the dye in solution, as demonstrated in FIGS. 3 and 4. Thus, the binding affinity of the test compound may be calculated as a function of its ability to displace the dye from the BIR3 domain, according to the following calculation:

$${K}_{relative} = \frac{{{Fraction}_{free}^{2} [ badan ]}_{total}}{( 1 - {Fraction}_{free} ) ( {[ AVPX ]}_{total} - {[ badan ]}_{total} {Fraction}_{free} )}$$

Details of a typical assay are set forth below.

Materials:

63 μM BIR3 in 50 mM Kphos buffer pH 7 100 mM NaCl2 mM DTT
   Four 0.5 ml aliquots of BIR3 stored at −70° C. and thawed over ice were used
43.8 μM AVPC-badan in H₂O; chilled to 4° C.
   absorbance at 387 nm=0.9205; ε₃₈₇ₙₘ=21000 M⁻¹ cm⁻¹
50 mM tetrapeptide solutions in H₂O; chilled to 4° C.
50 mM Kphos buffer pH 7 100 mM NaCl 2 mM DTT; chilled to 4° C.
H₂O (MilliQ purified); chilled to 4° C.
   Procedure

Stock solution of badan, BIR3, and buffer were mixed: 2.5 ml of badan, 1.75 ml BIR3, and 15.25 ml of buffer were mixed in a glass vial which had been chilled to 4° C. Added 390 μL of the stock solution to 50 wells in the pre-chilled 96 well plate (wells A1-E2).

Stock solution of badan and buffer were mixed: 150 μL badan and 1020 μL of buffer were mixed in a small glass vial (also chilled) and added to 3 wells on the plate in 390 μL aliquots (F1-F3).

The 96 well plate was stored over ice in an insulated bucket while the emission spectra of the samples were taken. Fifty μL of the appropriate test solution (or water, for the control experiments) was added with a micropipet, the solution mixed with a Pasteur pipet before adding the sample to the fluorescence cuvette. While one sample was being scanned, the cuvette from the previous scan was washed with EtOH and then next sample was prepared.

The PTI fluorometer settings were as follows:
λₑₓ=387 nm; the emission spectrum was scanned from 420-650 nm
slits=5 nm dispersion
PMT voltage=750 mV
   The scan was done in 1 nm increments and the integration time was 1 s.

Using the above assay, the inventors have screened a wide variety of peptides and peptide mimetics for their ability to bind to the BIR3 domain of XIAP. As an example, a tetrapeptide library was created, in which positions 1, 2 and 4 of the Smac tetrapeptide were substituted with other components. In one series of constructions, substitutions were as follows:
1. Position 1: XVPI (SEQ ID NO:9), where X=Serine, Glycine or Aminobutyric acid.
2. Position 2: AXPI (SEQ ID NO:10), where X=all twenty naturally occurring amino acids.
3. Position 4: AVPX (SEQ ID NO: 1), where X=all twenty naturally occurring amino acids.

Samples of results of the assay performed on members of the aforementioned group are shown in Table 1.

TABLE 1

| SEQ ID: | Sample | Intensity (470 nm) | Fraction_{free} | Kᵣₑₗₐₜᵢᵥₑ |
|---|---|---|---|---|
| 4 | AVPF | 16773 | 0.97410 | 31.5300 |
| 11 | AVPW | 23435 | 0.94176 | 23.1330 |
| 5 | ARPI | 29455 | 0.91253 | 4.3126 |
| 12 | ALPI | 38650 | 0.86789 | 3.5812 |
| 13 | AbuVPI | 34770 | 0.88673 | 3.0455 |
| 14 | AIPI | 44902 | 0.83754 | 2.6613 |
| 15 | AVPY | 39093 | 0.86574 | 2.5442 |
| 3 | AVPI | 54232 | 0.79224 | 2.5014 |
| 16 | AHPI | 41450 | 0.85430 | 2.2917 |
| 3 | AVPI | 26924 | 0.92482 | 2.2415 |

The tetrapeptides AVPF (SEQ ID NO:4), AIAY (SEQ ID NO:17) and AVAF (SEQ ID NO: 18) correspond in sequence to Drosophila homologs of Smac. Results showed that tetrapeptides containing these sequences bound strongly to BIR3 (AVPF shown in Table 1, other results not shown).

The most successful modification at position 2 was ARPI (SEQ ID NO:5). The positive charge on the arginine residue may have contact with the surrounding negatively-charged residues in the binding pocket, resulting in the strong binding observed with ARPI (SEQ ID NO:5).

As mentioned, a tetrapeptide library of position-4 modifications was created. Table 2 below sets forth binding constants obtained for each member of this library, as tested with the assay of the invention.

TABLE 2

| SEQ ID: | Tetrapeptide | K |
|---|---|---|
| 4 | AVPF | >20 |
| 3 | AVPI (std) | 4.2149 |
| 15 | AVPY | 1.1692 |
| 11 | AVPW | 1.0817 |
| 19 | AVPL | 0.34232 |
| 3 | AVPI | 0.29080 |
| 20 | AVPD | 0.17988 |
| 21 | AVPT | 0.14300 |
| 2 | AVPC | 0.10340 |
| 22 | AVPV | 0.10111 |
| 23 | AVPG | 0.089481 |
| 24 | AVPH | 0.075209 |
| 25 | AVPQ | 0.066115 |
| 26 | AVPA | 0.055180 |
| 27 | AVPM | 0.052881 |
| 28 | AVPE | 0.037089 |
| 29 | AVPN | 0.015724 |
| 30 | AVPS | 0.013041 |
| 31 | AVPP | 0.010695 |
| 32 | AVPK | 0.0070200 |
| 33 | AVPR | 0.0014831 |

Emission spectra of samples from this binding assay are shown in FIG. 4. As can be seen from FIG. 4 and the results set forth in Table 1 and Table 2, the tetrapeptide AVPF (SEQ ID NO: 4) bound strongly to the BIR3 domain, as evidenced by its ability to displace the AVP-dye. AVPW (SEQ ID NO11): and AVPY (SEQ ID NO:15) also showed binding at a strength equivalent to that of the naturally-occurring Smac peptide, AVPI (SEQ ID NO:3). By contrast, AVPK (SEQ ID NO:32) bound BIR3 only weakly.

In summary, the assay described herein has been demonstrated effective in identifying compounds that are capable of binding to the BIR3 domain of XIAP. Certain tetrapeptides with greater binding ability than the naturally-occurring Smac tetrapeptide have been identified. These tetrapeptides may be developed as therapeutic agents for the promotion of apoptosis in treatment of diseases or pathological conditions in which cell proliferation plays a role. The assay may be further used in high throughput screening of large panels of compounds generated by combinatorial chemistry or other avenues of rational drug design.

The following nonlimiting example is set forth to describe the invention in greater detail. The example contains data that replicate and supplement the data presented above. The example also describes additional tetrapeptide analogs, including N-methyl analogs and a dual substituted tetrapeptide, ARPF.

### EXAMPLE 1

### Molecular Targeting of Inhibitor of Apoptosis Proteins Based on Small Molecule Mimics of Natural Binding Partners

In this example, a fluorescence assay was used to test the binding of a library of tetrapeptides modeled on the Smac N-terminus to the surface pocket of the BIR3 region of XIAP. The results make it possible to parse the contribution of each residue of the tetrapeptide to the total binding energy of the interaction.

### Materials and Methods

Materials. Unless otherwise stated, materials were purchased from Aldrich Chemical Co. (Milwaukee, Wis.) or Fisher Scientific (Pittsburgh, Pa.) and used without further purification. Methylbenzhydrylamine (MBHA) solid-phase peptide synthesis resin, Rink amide resin, and 9-Fluorenylmethoxycarbonyl (Fmoc) protected amino acids were obtained from Advanced ChemTech (Louisville, Ky.) and NovaBiochem (San Diego, Calif.). 6-Bromoacetyl-2-dimethylaminonaphthalene (badan) dye was obtained from Molecular Probes (Eugene, Oreg.).

Synthesis of A VPC-badan. The peptide was synthesized by Fmoc protocol on MBHA resin. The MBHA resin was chosen because the protocol requires that the linkage to the solid support be stable under both acidic and basic conditions. The Ala-Val-Pro-Cys-NH₂ (AVPC; SEQ ID NO:2) peptide was synthesized using a trityl group to protect the cysteine thiol. The trityl group was removed by treatment with trifluoroacetic acid (TFA), and the cysteine was derivatized with badan in the presence of diisopropylethylamine (DIEA). The Fmoc group of the alanine was removed with piperidine and then cleavage from the resin was effected by treatment with anhydrous HF containing 10% v/v anisole as scavenger at 0° C. for 45 minutes. The labeled peptide was purified by HPLC on a Vydac C18 preparative column with gradient elution by solvents A (99% H₂O; 1% CH₃CN; 0.1% TFA) and B (90% CH₃CN; 10% H₂O; 0.1% TFA) and lyophilized to dryness prior to reconstitution in H₂O.

Synthesis of N-Fmoc-N-methyl-amino acids. N-methyl-amino acids were synthesized according to the methods of Freidinger et. al. (J. Org. Chem. 48: 77-81, 1983). The N-Fmoc-N-methyl-isoleucine and N-Fmoc-N-methyl phenylalanine were chromatographed over silica gel (5% methanol in chloroform as eluent); the N-Fmoc-N-methyl-valine was used without further purification.

Synthesis of Tetrapeptide Libraries. With the exception of the position one library and A(N-Me)VPI, all of the library molecules were synthesized on an Advanced ChemTech 396 MPS automated peptide synthesizer by Fmoc protocol on Rink amide resin (Chan & White (2000) Fmoc Solid Phase Synthesis, A Practical Approach; Oxford University Press, Oxford). For the AVPX (SEQ ID NO:1) and the AXPI (SEQ ID NO:10) libraries, the X positions were substituted with all twenty naturally occurring amino acids. The side chains of the amino acids that are sensitive to side reactions were protected as follows: cysteine, histidine, asparagine, and glutamine were protected using a trityl group; aspartic acid, glutamic acid, serine, threonine, and tyrosine were t-butyl protected; lysine and tryptophan were protected by Boc groups; and a pentamethyldihydrobenzofuran group was used to protect the arginine. After the alanine was added, deprotection and cleavage of the tetrapeptides from the resin was effected by adding 1 ml of a 95% TFA, 2.5% water, and 2.5% triisopropylsilane (TIS) solution to each well, and shaking for 1 hour. The cleavage solution was collected and a further 0.5 ml of the cleavage solution was added to each well and mixed for another hour. The combined cleavage solutions were added to 20 ml of water, lyophilized to dryness, then taken up in 5 ml of water before being filtered through syringe filters (0.2 μ) and lyophilized again.

The position one tetrapeptides and A(N-Me)VPI (SEQ ID NO:34) were synthesized on a hand shaker, also by Fmoc protocol on Rink amide resin. Cleavage and work up were done as described above. The presence of the desired tetrapeptide molecules was confirmed by mass spectroscopy.

The tetrapeptides were reconstituted in water and test solutions were made that were approximately 200 mM in the tetrapeptides. Exact concentrations were determined for 10 representative test solutions by ¹H-NMR using a dioxane solution of known concentration as an external reference. The concentrations of the other test solutions were taken to be the average value of the known solutions from the same library synthesis.

Expression and Purification of BIR3. Recombinant XIAP-BIR3 (residues 238-358) was overexpressed as a GST-fusion protein using pGEX-2T (Amersham Biosciences). The soluble fraction of the GST-BIR3 in the E. coli lysate was purified over a glutathione sepharose column, and further purified by anion exchange chromatography (Mono-Q, Amersham Biosciences). The fusion protein was cleaved by thrombin, and the GST portion was removed by the glutathione sepharose column. The BIR3 protein was further purified over a gel filtration column (Superdex 30, Amersham Biosciences).

Fluorescence Experiments. Luminescence spectra were recorded using a Photon Technologies, Inc. fluorometer with a Xe arc lamp and a PMT detector. The absorbance of all solutions was less than 0.2 at the excitation wavelength (387 nm). The buffer used in all of the fluorescence experiments was 50 mM potassium phosphate, 100 mM NaCl, 2 mM 1,4-dithio-DL-threitol (DTT), pH 7.

Determination of A VPC-badan binding constant to BIR3. 2 ml of a 2 μM AVPC-badan stock solution (buffer same as above) was titrated with a BIR3 stock solution from 0 to 10 μM in 15 μL increments. The dissociation constant for AVPC-badan and BIR3 was determined from the intensity observed at 470 μm after each addition of the protein.

Assay of Tetrapeptide Libraries. The samples were prepared in a 96 well plate lined with glass tubes, to prevent adsorption of the dye to plastic. The plate was stored on ice in the dark between measurements. A small volume cuvette, with a path length of 2 mm, was used to collect the emission spectra. 2.5 ml of a 44 μM aqueous solution of AVPC-badan, 1.75 ml of a 63 μM BIR3 solution, and 15.25 ml of buffer were mixed to give a stock solution which was 5.6 μM in both AVPC-badan and BIR3. 390 μL of this stock solution were added to 50 wells of the 96 well plate. 50 μL of the test tetrapeptide solutions were added and mixed immediately prior to taking the emission spectra. The final solutions were 5 μM in both badan and BIR3, and approximately 20-30 μM in the tetrapeptide solutions. 50 μL of water were added to three of the wells by way of controls, to determine the intensity observed when the AVPC-badan was bound to BIR3. 190 μL of AVPC-badan and 1020 μL of buffer were mixed and added to three wells in 390 μL aliquots. 50 μL of water was added to these wells, again as controls, to determine the intensity of the unbound dye. Equilibrium constants were determined by relating the observed intensity of the test solution at 470 nm to the average values obtained from the control experiments.

### Results

The binding of various tetrapeptide mimics to the BIR3 domain of XIAP was determined using a fluorescence-based competition assay. The assay is based on an environment-sensitive fluorogenic dye molecule, badan. Badan is a dye whose sensitivity to environmental changes has previously been used to probe protein binding interactions. A tetrapeptide based on the Smac binding motif, Ala-Val-Pro-Cys-NH₂ (AVPC; SEQ ID NO:2), was derivatized with the badan molecule to create a binding interaction with BIR3. When AVPC-badan binds to the surface groove of BIR3, changing the environment of the dye from water to the hydrophobic interior of the protein, the result is a large shift in both fluorescence maximum and intensity. The K_{D} for the AVPC-badan/BIR3 complex, as determined from a fluorescence titration, is 0.31±0.04 μM. The AVPC-badan can be displaced from the binding pocket of the protein by any competing molecule. As the dye is displaced from the binding pocket by the test molecule, the emission shifts back towards the aquated spectrum. Thus, the observed emission intensity of the dye can be related to the degree of displacement of AVPC-badan by the test molecules. This allows the most promising inhibitors to be quickly determined, and structural information about effective inhibitors can be incorporated into the design of candidates for the next round of testing.

Using the four N-terminal residues of Smac as a starting point, six libraries of related tetrapeptides were synthesized (Scheme 1) and evaluated in terms of their ability to displace AVPC-badan from the peptide binding groove on the surface of BIR3. The tetrapeptide libraries were designed to deconvolve the contribution of each amino acid to the binding of Smac to BIR3 (Scheme 1). The position one library only consisted of three members, reflecting the critical role that Ala1 plays in the recognition of the binding element by BIR3. The role of position three was explored using a tetrapeptide based on the N-terminal sequence of Reaper, one of the few natural binding partners without a proline in position three (Table 3). Libraries of positions two and four, over all twenty naturally occurring amino acids, were synthesized. The tetrapeptide ARPF (SEQ ID NO:35) was synthesized to investigate the possibility of additivity by modifying both positions simultaneously.

There are two bonds in the tetrapeptide that are vulnerable to proteolysis; the peptide bond between position one and position two, and the peptide bond between position three and four. One means of rendering these bonds more resistant to proteolysis is to replace the hydrogen on the amide with a methyl group. Several tetrapeptide homologs were synthesized with N-methyl amino acids to explore the effect such modifications have on the affinity of these compounds for BIR3.

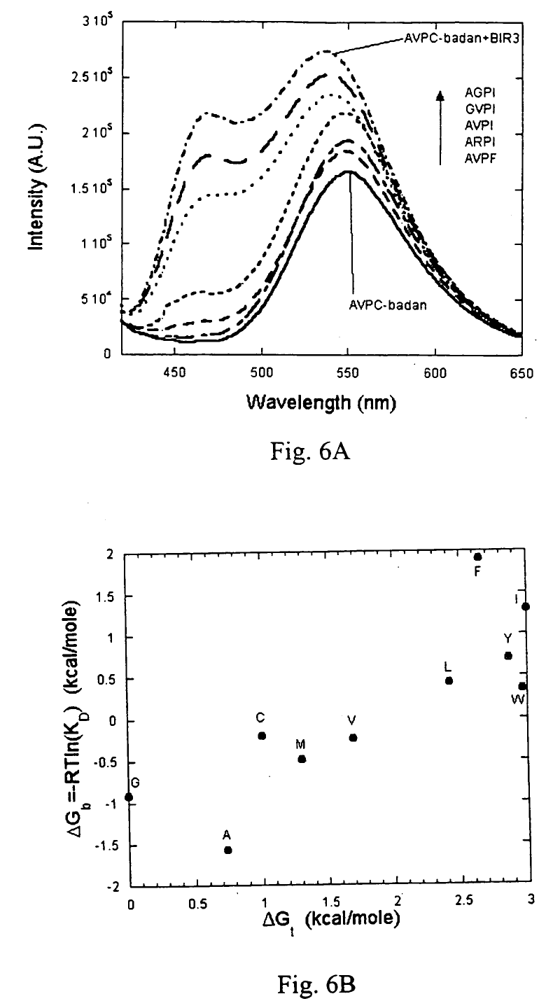

The dissociation constants (K_{D}) for the library members are listed in Table 4. The tetrapeptide mimics displace badan from BIR3 with varying facility (Table 4, FIG. 6A). The K_{D} values ranged from 0.02 μM to greater than 100 μM. The conservation of sequence of the binding motif observed across the range of protein binding partners suggests that nature has optimized the appropriate sequence to some extent, but the variety of tetrapeptides tested in this assay explores the specific contribution made at each position to the overall binding interaction.

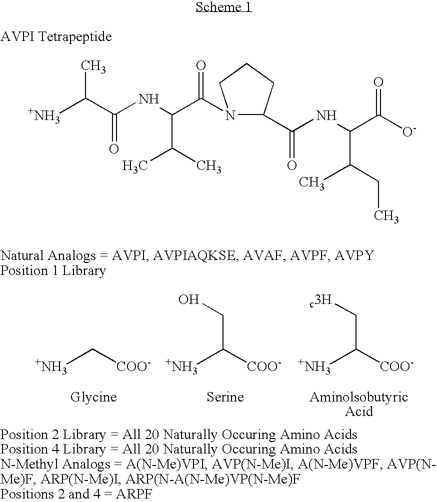

[image] TABLE 4
K_{D} for Tetrapeptide Homologs
(Numbers to the right of each sequence in parentheses are SEQ ID NOS)

| | K_{D} (μM) |
|---|---|
| Natural Analogs | |
| AVPI (3) | 0.48 |
| AVPIAQKSE (36) | 0.40 |
| AVAF (46) | 0.56 |
| AVPF (4) | 0.04 |
| AVPY (15) | 0.30 |
| Position 1 | |
| AbuVPI (13) | 0.24 |
| GVPI (6) | 9 |
| SVPI (47) | 27 |
| Position 2 | |
| ARPI (5) | 0.18 |
| ALPI (12) | 0.29 |
| AHPI (16) | 0.33 |
| AIPI (14) | 0.39 |
| AKPI (48) | 0.57 |
| AYPI (49) | 0.59 |
| ACPI (50) | 0.65 |
| AMPI (51) | 0.73 |
| AFPI (52) | 0.79 |
| AQPI (53) | 0.94 |
| AWPI (54) | 0.99 |
| ATPI (55) | 1.2 |
| ASPI (56) | 1.4 |
| ANPI (57) | 1.5 |
| AEPI (58) | 2.7 |
| AAPI (59) | 2.8 |
| ADPI (60) | 17 |
| AGPI (7) | 46 |
| APPI (61) | >100 |
| Position 4 | |
| AVPW (11) | 0.11 |
| AVPL (19) | 0.49 |
| AVPC (2) | 1.4 |
| AVPV (22) | 1.5 |
| AVPT (21) | 2.1 |
| AVPM (27) | 2.3 |
| AVPS (30) | 4.4 |
| AVPG (23) | 4.7 |
| AVPP (31) | 5.7 |
| AVPD (20) | 7.3 |
| AVPH (24) | 7.3 |
| AVPA (26) | 14 |
| AVPK (32) | 28 |
| AVPE (28) | 93 |
| AVPR (33) | >100 |
| AVPN (29) | >100 |
| AVPQ (25) | >100 |
| Positions 2 and 4 | |
| ARPF (35) | 0.02 |
| N-methyl Analogs | |
| ARP(N-Me)F (62) | 0.71 |
| AVP(N-Me)F (63) | 0.89 |
| A(N-Me)VPF (64) | 83 |
| A(N-Me)VP(N-Me)F(65) | 91 |
| AVP(N-Me)I (66) | 174 |
| ARP(N-Me)I (67) | 190 |
| A(N-Me)VPI (68) | 257 |

### Discussion

Residue 1

In previous studies, it was noted that mutations of the N-terminal amino acid of Smac completely abrogated the binding interaction between Smac and BIR3. The recognition between Smac and the surface groove of the BIR3 is based on a combination of eight intermolecular hydrogen bonds and van der Waals contacts. The necessity of the N-terminal alanine is obvious from the crystal structure. Ala1 donates three hydrogen bonds to nearby residues in the surface groove of BIR3, and its carbonyl group makes two additional contacts. The methyl group of Ala1 fits tightly into a hydrophobic pocket, and any modification of the alanine residue must be carefully designed to avoid steric hindrance in this pocket, or disruption of any of these essential hydrogen bonds. Although the next three residues contribute to the positioning of Ala1 in the binding pocket, their identity does not appear to be as critical as that of the Ala1.

The position one library members demonstrate how sensitive the binding interaction is to any modification at this position. Binding is greatly diminished with GVPI (SEQ ID NO:6), consistent with an earlier report, and SVPI (SEQ ID NO:47) is also a diminished binder, but a slight enhancement in binding was observed with the unnatural amino acid, aminoisobutyric acid (Abu).

Residue 3

AVAF (SEQ ID NO:46) has a binding affinity similar to that observed for the other natural analogs, AVPI (SEQ ID NO:3) and AVPIAQKSE (SEQ ID NO:36). However, this affinity is diminished by greater than a factor of ten relative to that observed for the AVPF (SEQ ID NO:4) tetrapeptide from the position two library. Previous studies have also noted a decrease in binding affinity when the proline is replaced by alanine. Based on that observation, and the relative homogeneity observed in the natural binding partners at position three (Table 3), it would seem that replacing the proline will diminish the binding affinity of the test tetrapeptide.

Residue 2

As stated earlier, nature has already optimized the appropriate sequence to some extent. However, the position two library gives some surprising results. The high affinity of tetrapeptides such as ARPI (SEQ ID NO:5) and AHPI (SEQ ID NO:16) relative to the natural sequence of AVPI (SEQ ID NO: 3) would seem to indicate that positive charge at position two would increase the binding affinity of the peptide. This is not an unexpected result given the negatively charged residues that line the binding pocket of BIR3. Nonetheless, none of the natural binding partners of IAP listed in Table 3 has positively charged residues at position two. All the natural LAP interacting motifs that have been observed so far all contain b-branched amino acids at position two, such as valine, threonine, and isoleucine (Table 3). This result indicates that the natural sequence can be improved upon, and gives a basis for the structural design of the next set of potential binding partners.

Residue 4

The X-ray structure of Smac binding to BIR3 indicates that there are no intermolecular hydrogen bonds to residue 4, and, of the four residues of the binding motif, residue 4 is the least sterically hindered. This would seem to make position four least sensitive to modification. Indeed, the K_{D} that is observed for the AVPC (SEQ ID NO: 2) tetrapeptide (Table 4) is greater than that of the AVPC-badan, which indicates that binding is slightly enhanced by the presence of the dye. However, a much wider range of K_{D}s is observed for the position four library than for the position two library. Although modification at this position can lead to the greatest enhancement in binding affinity that is observed, it can also essentially destroy the binding interaction.

The AVPF (SEQ ID NO:4) tetrapeptide was by far the most strongly binding library member, closely followed by AVPW (SEQ ID NO:11). AVPY (SEQ ID NO:15) was also determined to have a binding affinity slightly greater than the natural analog, AVPI (SEQ ID NO:3). These results indicate that an aromatic group side chain on the amino acid at position four substantially enhances the binding affinity of the tetrapeptide for BIR3. This result is consistent with phylogenic data: other proteins that interact with LAPs have phenylalanine or tyrosine at position four (Table 3).

When high affinity substitutions at position two and four were probed simultaneously using the ARPF tetrapeptide, the effects were found to be additive. Consequently, the detrimental effect on binding affinity observed with the N-methylated tetrapeptides could be somewhat counteracted by the increased affinity gained from the appropriate choice of amino acid.

N-methyl Analogs

N-methylation at the peptide bond between residues 1 and 2 disrupts a structurally defined hydrogen bond, and has a correspondingly large effect on binding. By contrast, N-methylation of residue 4 has a much smaller effect, consistent with structural data, which show no hydrogen bond to this amide. From a standpoint of molecular design, this relieves an important design constraint. Consideration of side chain contributions to the free energy of binding, ΔG_{b}, using the free energy of transfer from ethanol to water, ΔGₜ (EtOH—H₂O), to approximate the energy contribution of the side chain for hydrophobic amino acids, follows a clear general trend. More hydrophobic amino acids clearly bind more strongly, as indicated in FIG. 6B. The obvious correlation indicates that there is little specificity of interaction, but also suggests that the full hydrophobic effect is not realized. For example, the ΔGₜ of W is greater than that of F, but the ΔG_{b} of AVPF (SEQ ID NO:4) is greater than that of AVPW (SEQ ID NO:11). A more detailed analysis can be obtained by modeling the various peptides onto the known structure and determining the solvent exposed surface area within the model.

This invention is not limited to the embodiments described and exemplified above, but is capable of variation and modification within the scope of the appended claims.

1. An assay for determining if a test agent is capable of binding a BIR domain of an Inhibitor of Apoptosis Protein (IAP), comprising the steps of:
a) providing a detectably labeled peptide or peptidomimetic compound that binds to a BIR domain of the IAP, wherein the compound has a formula:
R₁—R₂—R₃—R₄
wherein R₁ is A or a mimetic of A;
R₂ is V, T or I or a mimetic of V, T or I;
R₃ is P or A or a mimetic of P or A; and
R₄ is any amino acid or a mimetic thereof and the detectable label is associated with R₄;
wherein at least one measurable feature of the detectable label changes as a function of the labeled compound being either bound to the IAP or free in solution;
b) contacting the IAP with the labeled compound under conditions enabling binding of the labeled compound to the IAP, thereby forming a labeled compound/IAP complex having the measurable feature;
c) contacting the labeled compound/IAP complex with the test agent; and
d) measuring displacement of the labeled compound from the labeled compound/IAP complex, if any, by the test agent, by measuring the change in the measurable feature of the labeled compound, thereby determining if the test agent is capable of binding to the IAP.

2. The assay of claim 1, wherein the labeled compound is a peptide AVPX, wherein X is any amino acid

3. The assay of claim 1, wherein the label is a fluorigenic dye.

4. The assay of claim 3, wherein the labeled compound is a peptide AVPX, wherein X is any amino acid and is directly or indirectly linked to the fluorigenic dye.

5. The assay of claim 4, wherein the labeled compound is AVPC-badan dye.

6. The assay of claim 1, wherein the BIR domain is a BIR3 domain or a BIR2 domain.

7. The assay of claim 1, wherein the BIR domain is provided as part of an intact IAP.

8. A detectably labeled compound for performing a assay to determine if a test agent is capable of binding a BIR domain of an Inhibitor of Apoptosis Protein (IAP), wherein the compound has a formula:
R₁—R₂—R₃—R₄
wherein R₁ is A or a mimetic of A;
R₂ is V, T or I or a mimetic of V, T or I;
R₃ is P or A or a mimetic of P or A; and
R₄ is any amino acid or a mimetic thereof and the detectable label is associated with R₄;
wherein at least one measurable feature of the detectable label changes as a function of the labeled compound being either bound to the IAP or free in solution.

9. The labeled compound of claim 8, comprising a peptide AVPX, wherein X is any amino acid

10. The compound of claim 8, wherein the label is a fluorigenic dye.

11. The compound of claim 10, comprising a peptide AVPX, wherein X is any amino acid and is directly or indirectly linked to the fluorigenic dye.

12. The compound of claim 11, which is AVPC-badan dye.

13. An assay for determining if a test compound is capable of binding a BIR3 domain of an Inhibitor of Apoptosis Protein (LAP), comprising the steps of:
a) providing a labeled mimetic of an AVPI tetrapeptide that binds to the BIR3 domain, wherein at least one measurable feature of the label changes as a function of the mimetic being bound to the IAP or free in solution;
b) contacting the IAP with the labeled mimetic under conditions enabling binding of the mimetic to the IAP, thereby forming an IAP/labeled mimetic complex having the measurable feature;
c) contacting the IAP/labeled mimetic complex with the test compound; and
d) measuring displacement of the labeled mimetic from the IAP/labeled mimetic complex, if any, by the test compound, by measuring the change in the measurable feature of the labeled mimetic, thereby determining if the test compound is capable of binding to the IAP.

14. The assay of claim 13, wherein the labeled mimetic is AVPX, wherein X is directly or indirectly linked to a fluorigenic dye.

15. The assay of claim 13, wherein the labeled mimetic is AVPC-badan dye.

16. The assay of claim 1, wherein the LAP is substituted with a portion of the IAP comprising the BIR3 domain.


SEQUENCE LISTING (68 sequences)

SEQ ID NO: 1 - protein, 4 aa, Artificial Sequence
  Synthetic tetrapeptide sequence
AVPX

SEQ ID NO: 2 - protein, 4 aa, Artificial Sequence
  Synthetic tetrapeptide sequence
AVPC

SEQ ID NO: 3 - protein, 4 aa, Artificial Sequence
  Synthetic tetrapeptide sequence
AVPI

SEQ ID NO: 4 - protein, 4 aa, Artificial Sequence
  Synthetic tetrapeptide sequence
AVPF

SEQ ID NO: 5 - protein, 4 aa, Artificial Sequence
  Synthetic tetrapeptide sequence
ARPI

SEQ ID NO: 6 - protein, 4 aa, Artificial Sequence
  Synthetic tetrapeptide sequence
GVPI

SEQ ID NO: 7 - protein, 4 aa, Artificial Sequence
  Synthetic tetrapeptide sequence
AGPI

SEQ ID NO: 8 - protein, 4 aa, Artificial Sequence
  Synthetic tetrapeptide sequence
AXXX

SEQ ID NO: 9 - protein, 4 aa, Artificial Sequence
  Synthetic tetrapeptide sequence
XVPI

SEQ ID NO: 10 - protein, 4 aa, Artificial Sequence
  Synthetic tetrapeptide sequence
AXPI

SEQ ID NO: 11 - protein, 4 aa, Artificial Sequence
  Synthetic tetrapeptide sequence
AVPW

SEQ ID NO: 12 - protein, 4 aa, Artificial Sequence
  Synthetic tetrapeptide sequence
ALPI

SEQ ID NO: 13 - protein, 4 aa, Artificial Sequence
  Synthetic tetrapeptide sequence
XVPI

SEQ ID NO: 14 - protein, 4 aa, Artificial Sequence
  Synthetic tetrapeptide sequence
AIPI

SEQ ID NO: 15 - protein, 4 aa, Artificial Sequence
  Synthetic tetrapeptide sequence
AVPY

SEQ ID NO: 16 - protein, 4 aa, Artificial Sequence
  Synthetic tetrapeptide sequence
AHPI

SEQ ID NO: 17 - protein, 4 aa, Artificial Sequence
  Synthetic tetrapeptide sequence
AIAY

SEQ ID NO: 18 - protein, 4 aa, Artificial Sequence
  Synthetic tetrapeptide sequence
AVAF

SEQ ID NO: 19 - protein, 4 aa, Artificial Sequence
  Synthetic tetrapeptide sequence
AVPL

SEQ ID NO: 20 - protein, 4 aa, Artificial Sequence
  Synthetic tetrapeptide sequence
AVPD

SEQ ID NO: 21 - protein, 4 aa, Artificial Sequence
  Synthetic tetrapeptide sequence
AVPT

SEQ ID NO: 22 - protein, 4 aa, Artificial Sequence
  Synthetic tetrapeptide sequence
AVPV

SEQ ID NO: 23 - protein, 4 aa, Artificial Sequence
  Synthetic tetrapeptide sequence
AVPG

SEQ ID NO: 24 - protein, 4 aa, Artificial Sequence
  Synthetic tetrapeptide sequence
AVPH

SEQ ID NO: 25 - protein, 4 aa, Artificial Sequence
  Synthetic tetrapeptide sequence
AVPQ

SEQ ID NO: 26 - protein, 4 aa, Artificial Sequence
  Synthetic tetrapeptide sequence
AVPA

SEQ ID NO: 27 - protein, 4 aa, Artificial Sequence
  Synthetic tetrapeptide sequence
AVPM

SEQ ID NO: 28 - protein, 4 aa, Artificial Sequence
  Synthetic tetrapeptide sequence
AVPE

SEQ ID NO: 29 - protein, 4 aa, Artificial Sequence
  Synthetic tetrapeptide sequence
AVPN

SEQ ID NO: 30 - protein, 4 aa, Artificial Sequence
  Synthetic tetrapeptide sequence
AVPS

SEQ ID NO: 31 - protein, 4 aa, Artificial Sequence
  Synthetic tetrapeptide sequence
AVPP

SEQ ID NO: 32 - protein, 4 aa, Artificial Sequence
  Synthetic tetrapeptide sequence
AVPK

SEQ ID NO: 33 - protein, 4 aa, Artificial Sequence
  Synthetic tetrapeptide sequence
AVPR

SEQ ID NO: 34 - protein, 4 aa, Artificial Sequence
  Synthetic tetrapeptide sequence
AVPI

SEQ ID NO: 35 - protein, 4 aa, Artificial Sequence
  Synthetic tetrapeptide sequence
ARPF

SEQ ID NO: 36 - protein, 9 aa, Artificial Sequence
  Synthetic heptapeptide sequence
AVPIAQKSE

SEQ ID NO: 37 - protein, 9 aa, Artificial Sequence
  Synthetic heptapeptide sequence
AVPSPPPAS

SEQ ID NO: 38 - protein, 9 aa, Artificial Sequence
  Synthetic heptapeptide sequence
AVAFYIPDQ

SEQ ID NO: 39 - protein, 9 aa, Artificial Sequence
  Synthetic heptapeptide sequence
AIAYFLPDQ

SEQ ID NO: 40 - protein, 9 aa, Artificial Sequence
  Synthetic heptapeptide sequence
AVPFYLPEG

SEQ ID NO: 41 - protein, 9 aa, Artificial Sequence
  Synthetic heptapeptide sequence
ATPFQEGLR

SEQ ID NO: 42 - protein, 9 aa, Artificial Sequence
  Synthetic heptapeptide sequence
AVPYQEGPR

SEQ ID NO: 43 - protein, 9 aa, Artificial Sequence
  Synthetic heptapeptide sequence
ATPVFSGEG

SEQ ID NO: 44 - protein, 9 aa, Artificial Sequence
  Synthetic heptapeptide sequence
SGPINDTDA

SEQ ID NO: 45 - protein, 9 aa, Artificial Sequence
  Synthetic heptapeptide sequence
SGVDDDMAC

SEQ ID NO: 46 - protein, 4 aa, Artificial Sequence
  Synthetic tetrapeptide sequence
AVAF

SEQ ID NO: 47 - protein, 4 aa, Artificial Sequence
  Synthetic tetrapeptide sequence
SVPI

SEQ ID NO: 48 - protein, 4 aa, Artificial Sequence
  Synthetic tetrapeptide sequence
AKPI

SEQ ID NO: 49 - protein, 4 aa, Artificial Sequence
  Synthetic tetrapeptide sequence
AYPI

SEQ ID NO: 50 - protein, 4 aa, Artificial Sequence
  Synthetic tetrapeptide sequence
ACPI

SEQ ID NO: 51 - protein, 4 aa, Artificial Sequence
  Synthetic tetrapeptide sequence
AMPI

SEQ ID NO: 52 - protein, 4 aa, Artificial Sequence
  Synthetic tetrapeptide sequence
AFPI

SEQ ID NO: 53 - protein, 4 aa, Artificial Sequence
  Synthetic tetrapeptide sequence
AQPI

SEQ ID NO: 54 - protein, 4 aa, Artificial Sequence
  Synthetic tetrapeptide sequence
AWPI

SEQ ID NO: 55 - protein, 4 aa, Artificial Sequence
  Synthetic tetrapeptide sequence
ATPI

SEQ ID NO: 56 - protein, 4 aa, Artificial Sequence
  Synthetic tetrapeptide sequence
ASPI

SEQ ID NO: 57 - protein, 4 aa, Artificial Sequence
  Synthetic tetrapeptide sequence
ANPI

SEQ ID NO: 58 - protein, 4 aa, Artificial Sequence
  Synthetic tetrapeptide sequence
AEPI

SEQ ID NO: 59 - protein, 4 aa, Artificial Sequence
  Synthetic tetrapeptide sequence
AAPI

SEQ ID NO: 60 - protein, 4 aa, Artificial Sequence
  Synthetic tetrapeptide sequence
ADPI

SEQ ID NO: 61 - protein, 4 aa, Artificial Sequence
  Synthetic tetrapeptide sequence
APPI

SEQ ID NO: 62 - protein, 4 aa, Artificial Sequence
  Synthetic tetrapeptide sequence
ARPF

SEQ ID NO: 63 - protein, 4 aa, Artificial Sequence
  Synthetic tetrapeptide sequence
AVPF

SEQ ID NO: 64 - protein, 4 aa, Artificial Sequence
  Synthetic tetrapeptide sequence
AVPF

SEQ ID NO: 65 - protein, 4 aa, Artificial Sequence
  Synthetic tetrapeptide sequence
AVPF

SEQ ID NO: 66 - protein, 4 aa, Artificial Sequence
  Synthetic tetrapeptide sequence
AVPI

SEQ ID NO: 67 - protein, 4 aa, Artificial Sequence
  Synthetic tetrapeptide sequence
ARPI

SEQ ID NO: 68 - protein, 4 aa, Artificial Sequence
  Synthetic tetrapeptide sequence
AVPI